(19) United States
(12) Patent Application Publication
(10) Pub. No.: US 2008/0312607 A1
(43) Pub. Date: Dec. 18, 2008

(54) Polypropylene Hollow Barrel with Sliding Coated Rubber Piston

(75) Inventors: Yves A. Delmotte, Neufmaison (BE); Agneta Blom, Wauthier-Braine (BE)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., Deerfield, IL (US); BAXTER HEALTHCARE S.A., Wallisellen (CH)

(21) Appl. No.: 11/915,787

(22) PCT Filed: May 12, 2006
(86) PCT No.: PCT/EP2006/004494

§ 371 Date: Jul. 2, 2008

Related U.S. Application Data
(60) Provisional application No. 60/685,830, filed on May 31, 2005.

(51) Int. Cl. A61M 5/315

(52) U.S. Cl. 604/230

(57) ABSTRACT

The present invention relates to a device comprising a piston sliding in a body like, for example, a syringe.

The present invention relates to a device comprising a piston sliding in a body like, for example, a syringe.

In most of commercial syringes, the piston sliding in the hollow barrel of a syringe body is made of a resilient material, such as rubber or thermoplastic elastomer, to absorb the irregularity in the shape of the syringe body. In order to allow the sliding and ensure that the syringe does not become leaky when pressure is applied, the sliding piston is coated with a silicone lubricant. One disadvantage of the use of silicone coated pistons is that that the silicone oils contaminate the content of the syringe body, e.g. a liquid medicament to be applied with the syringe.

In order to avoid such effect, laminated pistons were developed and disclosed in the prior art. Those pistons are of a silicone-free type in which it is not necessary to coat the sliding portion with a silicone oil layer as a lubricant.

In U.S. Pat. No. 6,090,081, pistons (rubber stoppers) are described which are capable of satisfying both the sealing property and slidable property without using silicone oils and having high sanitary and safety property. Such pistons are coated with a tetrafluoroethylene-ethylene copolymer resin as disclosed in Japanese Patent Laid-Open Publication No. 139668/1987, or with a polytetrafluoroethylene resin film, as disclosed in Japanese Patent Laid-Open Publication No. 97173/1988. The content of the said US patent and the Japanese Patent Publications are incorporated herein by reference.

Efforts were made to develop new materials for the body of the syringes which can be combined with the laminated pistons.

It is the merit of the present invention that it was surprisingly found that it is possible to combine certain pistons with certain coating with conventional hollow barrel bodies made of polypropylene in order to obtain a device fulfilling all functional, sanitary and regulatory requirements for use for medical purposes, like air and water tightness and required sliding forces.

In one embodiment of the invention, the surface of the resilient piston is coated with a laminated layer of polytetrafluoroethylene resin film, in another embodiment the surface of the resilient piston is coated with a laminated layer tetrafluoroethylene-ethylene resin film. The coating can be performed as described in U.S. Pat. No. 6,090,081 and the Japanese Patent Laid-Open Publication No. 139668/1987, or Japanese Patent Laid-Open Publication No. 97173/1988. In a further embodiment of the present invention, a tetrafluoroethylene polymer coated piston Flurotec commercially available from West Pharmaceutical-Daikyo as specified in Example 1 is used.

A hollow barrel polypropylene body according to the present invention may be any body made of polypropylene which is a hollow barrel intended for use in combination with a sliding piston, e.g. conventional syringe bodies or the like. It is within the ordinary skill of a worker in the field to be capable to combine a piston with a certain design with the appropriate hollow barrel in order to achieve the functional requirements such as air and water tightness and requiring a sliding force that fulfills the acceptance criteria of regulatory authorities.

Therefore, the invention concerns a device comprising a combination of a polypropylene body with a laminated piston defined above as possible embodiment of the invention.

The device exemplified in detail in the following example shall be another embodiment of the invention. However, the examples shall illustrate the invention and not be used to limit the scope of the teaching given herein.

### EXAMPLES

### Example 1

### TRICOS-Fluoro-Device

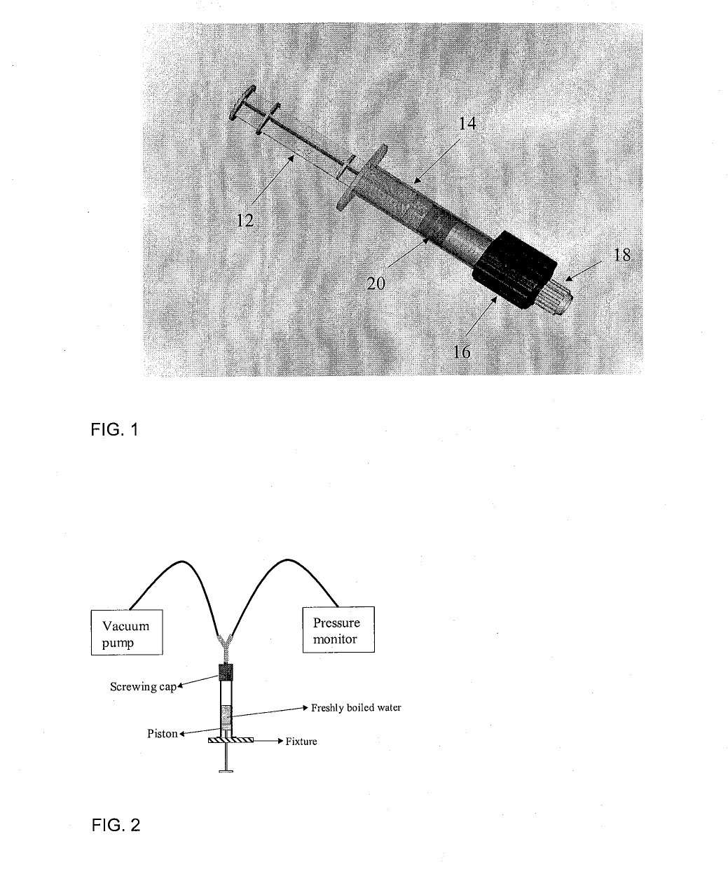

In a TRICOS-device (see FIG. 1) constituted of plunger, a body syringe, a screwing top and a luer cap as described in PCT patent publication number WO2004032808, the conventional rubber piston is replaced by 5 ml piston made of Butyl rubber coated with tetrafluoroethylene polymer resin as per BP 3P01020 obtained from West Pharmaceutical-Daikyo. Such amended TRICOS-device is named TRICOS-Fluoro-Device.

The different components of one embodiment of the TRICOS-Fluoro-Device are described in detail in the following table:

| | | Reference | |
|---|---|---|---|
| Name | Description | number | Lot number |
| Screwing | Molded part made of 96% | RE REF #195 | 10030F0020 |
| top | natural polypropylene | | |
| | (Grade: HD810MO) and | | |
| | 4% of Blue concentrate | | |
| | Polybatch P45056 as per | | |
| | BP Ind.01.10.001-D - | | |
| | Bouchon | | |
| Luer cap | Molded part made of PL | 20007803 | 10352601 |
| | 1747 as per BP PF0470 | | |
| Body | Molded part made of | RE REF #194 | 10030F0022 |
| syringe | natural polypropylene | | |
| | (Grade: HD810MO) as per | | |
| | BP Ind.01.10.001-D- Corps | | |
| | de seringue | | |
| Plunger | Molded part made of | RE REF #196 | 10030F0021 |
| | natural polypropylene | | |
| | (Grade: HD810MO) as per | | |
| | BP Ind.01.10.001- C- | | |
| | Piston de seringue | | |
| Piston | 5 ml piston made of Butyl | 5 ml Piston | 030110 |
| | rubber coated with | FR2-2RS- | |
| | tetrafluoroethylene | formulation: | |
| | polymer resin as per BP | D21-6-1 | |
| | 3P01020 | | |
| | obtained from West | | |
| | Pharmaceutical-Daikyo. | | |

The use of a tetrafluoroethylene polymer coated piston from Daikyo with the polypropylene syringe body, presents a lot of advantages for the development of the final product, as it does not require the use of silicone oil to facilitate the sliding of the piston inside of the syringe body. This is a tremendous advantage from a regulatory but also manufacturing point of view: easy to store, does not stick, inexpensive process and equipment, no transfer of the silicone oil to the granules of calcium phosphate.

### Further Embodiments of TRICOS-Fluoro-Device

“5 ml, 10 ml and 20 ml” tetrafluoroethylene polymer coated pistons (i.e pistons foreseen for conventional 5 ml, 10 ml, and 20 ml syringes, respectively) from Daikyo were successfully used with 3.5 ml, 7.0 ml and 17 ml TRICOS syringes (design History file: 001-DHF-NIV). Of course, it is understood that also pistons of other size could be used with the appropriate hollow barrel (TRICOS syringe) to produce a functional TRICOS-Fluoro-Device.

### Example 2

### Tests

Tests were performed to evaluate if the tetrafluoroethylene polymer resin coated piston can fulfill the acceptance criteria of the standards applying for syringe like container made of polypropylene HD810MO.

These standards are applied for commercial syringe made of polypropylene with a piston that is siliconized.

### 1. Air Leakage Between the Piston and the Inner Wall of the Syringe Body During Aspiration, and for Separation of Piston and Plunger as per ISO 7886-1 (Annex B)

This test challenges the ability of the syringe like container to resist to leakage and piston detachment from the plunger under negative pressure. This test is an attribute test based on the ISO 7886-1, Annex B of the norm.

A pass or fail determination was made based on a visual observation for replacing bubbles and piston detachment.

Protocol of test is described in EXAMPLE 3.

### Test Criteria:

No leak at piston is accepted and no piston detachment is accepted.

The pressure may not increase during the 60's test under vacuum

### Test Results:

75 non-sterile units and 75 sterile units were tested.

All units passed successfully the piston detachment test and no increase in pressure during the 60 seconds of vacuum was observed for any of the units tested. No piston leak was detected.

### Conclusions:

All tested units passed successfully test “Air leakage past piston during aspiration, and for separation of piston and plunger as per ISO 7886-1 (annex B of the norm)” and by that it can be stated with 95% confidence that there is less than 3.916% defective units.

### 2. Piston Pull-Out Test

This test challenges the ability of the piston to remain engaged with the plunger when exposed to a potential pull out force. A pass or fail determination was made.

Protocol of test is described in EXAMPLE 4.

### Test Criteria:

No piston detachment from the plunger is accepted

### Test Results:

75 non sterile units and 75 sterile units were tested.

All units passed successfully the test.

### Conclusions:

All tested units passed successfully “Piston pull-out test” and by that it can be stated with 95% confidence that there are less than 3.916% defective units as per test.

### 3. Piston Removal Force

This test challenges the ability of the piston/plunger to remain inserted into the body syringe when exposed to a potential pull out force. The force needed to remove the piston/plunger from the body syringe was measured thanks to a tensile machine and the maximum pull out force has to be higher than 29 N (precision movement sustained male—DEF STAN 00-25—part 3) and it is preferable that the maximum pull force is higher than 59 N (precision movement momentary male—DEF STAN 00-25—part 3).

Protocol of test is described in EXAMPLE 5.

### Test Results:

50 non-sterile units and 50 sterile units were tested. For both the sterile and the non-sterile units there were 3 units where the part of the plunger attached to the tensile machine broke before the plunger was removed. This means that the actual force needed to remove the plunger is above the value registered.

Peak force (N)

| | Non-sterile units | Sterile units |
|---|---|---|
| Average: | 198.4 | 178.2 |
| Min: | 112.1 | 118.3 |
| Max: | 281.1 | 233.2 |
| Standard Deviation: | 42.96 | 28.19 |

### Conclusions:

All tested units successfully passed the test, and it can be stated with 95% confidence that at least 99% of the units of an equal production, when tested according to test, will result in a peak force above 75.8 N for the non-sterile samples and above 97.8 N for the sterile samples.

### 4. Liquid Leakage at Syringe Piston Under Compression

The test challenges the ability of the syringe piston to resist leakage under axial pressure. This test is based on the ISO 7886-1.

A pass or fail determination was made.

Protocol of test is described in EXAMPLE 6.

### Test Criteria:

No leak is accepted

### Test Results:

75 non sterile units and 75 sterile units were tested.

No leak was detected for any of the units tested.

### Conclusions:

All test units passed successfully test “Liquid leakage at syringe piston under compression” and by that it can be stated with 95% confidence that there is less than 3.916% defective units.

### 5. Forces Required to Operate the Plunger

The test purpose is to measure the force, which is required to initiate the movement of the plunger inside of the syringe body. This test is based on the ISO 7886-1:1993 annex G

### Test Criteria:

In ISO 7886-1:1993 annex G there is no strict requirement on the force required to initiate the movement of the plunger, but a proposed value of <25 N is given.

It is known in the art that a piston cannot slide into the syringe body without coating with silicone oil.

### Test Results:

Protocol of test is described in EXAMPLE 7.

50 sterile units were tested.

| | Sterile units |
|---|---|
| | Initial force |
| | (N) |
| Average: | 18.43 |
| Min: | 12.12 |
| Max: | 22.95 |
| Standard Deviation: | 2.61 |

### Conclusions:

The force needed to initiate the movement of the plunger is below the proposed limit of 25 N for all units tested and it can be stated with 95% confidence that at least 99% of the units of an equal production when tested according to the above test, will result in an initial force to move the plunger below 25.9 N. These results are acceptable since there is no difficulty to move the piston at the forces obtained in this study

### 6. Check the Dimensions of the Pistons as per Blueprint Provided by Daikyo.

The tests are performed before sterilization and after beta sterilization at a dose of 50 kGy onto the overall dimensions of the TRICOS devices.

This test is important to show that the tetrafluoroethylene polymer resin coated piston from Daikyo keeps its dimensions after sterilization and therefore the its functionality when mounted in the TRICOS device as shown in tests 1 to 4

### Samples and Raw Material:

### Piston:

Traceability:
Production code: 5 ml Piston FR2-2RS from Daikyo
Formulation: D21-6-1
Lot No: 030110

Description:
Part made by Daikyo Seiko, LTD and supplied by West Pharmaceutical.
Butyl rubber part coated with Fluoro resin.

### Sample Preparation:

25 pistons were tested as received by the West supplier, while 25 other pistons were packed into an HDPE overpouch and sent to Ionisos for beta sterilization at 50 kGy before dimensional test.

Test description: visual inspection with a calibrated caliper

Performance: All 50 pistons were inspected. The 25 pistons for sterilization were inspected both before and after sterilization.

Result: No defective units were observed.

### Dimensional Check

Performance: 25 sterile and 25 non-sterile pistons were measured as per the attached blueprint. A letter as indicated on the blueprint identified each dimension.

### Result on Non-Sterile Units:

Dimension (mm)

| Unit # | A | B | C | D |
|---|---|---|---|---|
| 1 | 12.6 | 12.05 | 12.47 | 10.82 |
| 2 | 12.67 | 12.14 | 12.48 | 10.8 |
| 3 | 12.66 | 12.09 | 12.47 | 10.94 |
| 4 | 12.64 | 12.09 | 12.55 | 11.02 |
| 5 | 12.65 | 12.1 | 12.47 | 11.07 |
| 6 | 12.66 | 12.12 | 12.5 | 11.03 |
| 7 | 12.67 | 12.09 | 12.47 | 10.93 |
| 8 | 12.69 | 12.08 | 12.59 | 10.86 |
| 9 | 12.67 | 12.1 | 12.48 | 10.95 |
| 10 | 12.64 | 12.11 | 12.49 | 10.88 |
| 11 | 12.67 | 12.1 | 12.55 | 10.86 |
| 12 | 12.67 | 12.14 | 12.54 | 10.91 |
| 13 | 12.68 | 12.09 | 12.5 | 10.9 |
| 14 | 12.67 | 12.11 | 12.53 | 10.89 |
| 15 | 12.67 | 12.13 | 12.53 | 10.98 |
| 16 | 12.69 | 12.09 | 12.52 | 10.99 |
| 17 | 12.67 | 12.1 | 12.57 | 10.88 |
| 18 | 12.68 | 12.12 | 12.47 | 10.95 |
| 19 | 12.69 | 12.14 | 12.51 | 10.99 |
| 20 | 12.69 | 12.14 | 12.58 | 10.89 |
| 21 | 12.69 | 12.14 | 12.46 | 10.85 |
| 22 | 12.69 | 12.13 | 12.55 | 10.89 |
| 23 | 12.69 | 12.11 | 12.56 | 11.02 |
| 24 | 12.68 | 12.14 | 12.53 | 10.93 |
| 25 | 12.67 | 12.14 | 12.54 | 10.95 |
| Average | 12.67 | 12.11 | 12.52 | 10.93 |
| Stdev | 0.0208 | 0.0243 | 0.0394 | 0.0686 |

A, B, C and D are described on the attached drawing

### Sterile Units:

Dimension (mm)

| Unit # | A | B | C | D |
|---|---|---|---|---|
| 1 | 12.68 | 12.09 | 12.49 | 10.87 |
| 2 | 12.67 | 12.12 | 12.49 | 11.01 |
| 3 | 12.68 | 12.12 | 12.5 | 10.99 |
| 4 | 12.69 | 12.12 | 12.56 | 10.99 |
| 5 | 12.69 | 12.13 | 12.51 | 11.04 |
| 6 | 12.66 | 12.12 | 12.5 | 10.98 |
| 7 | 12.67 | 12.09 | 12.5 | 11 |
| 8 | 12.68 | 12.12 | 12.52 | 11 |
| 9 | 12.68 | 12.14 | 12.5 | 10.99 |
| 10 | 12.69 | 12.11 | 12.51 | 10.99 |
| 11 | 12.67 | 12.11 | 12.49 | 10.98 |
| 12 | 12.67 | 12.14 | 12.49 | 10.91 |
| 13 | 12.66 | 12.07 | 12.52 | 11.01 |
| 14 | 12.67 | 12.14 | 12.52 | 10.96 |
| 15 | 12.64 | 12.1 | 12.5 | 10.99 |
| 16 | 12.66 | 12.13 | 12.46 | 11.01 |
| 17 | 12.64 | 12.13 | 12.47 | 10.99 |
| 18 | 12.66 | 12.09 | 12.52 | 11 |
| 19 | 12.68 | 12.11 | 12.54 | 10.96 |
| 20 | 12.69 | 12.13 | 12.49 | 10.98 |
| 21 | 12.69 | 12.14 | 12.57 | 10.99 |
| 22 | 12.68 | 12.14 | 12.49 | 10.97 |
| 23 | 12.67 | 12.12 | 12.55 | 11.02 |
| 24 | 12.64 | 12.08 | 12.48 | 11.02 |
| 25 | 12.66 | 12.12 | 12.5 | 10.99 |
| Average | 12.67 | 12.12 | 12.51 | 10.99 |
| Stdev | 0.0155 | 0.0200 | 0.0264 | 0.0345 |

A, B, C and D are described on the attached drawing

### Conclusions:

All piston measurements performed were within the limits.

No significant differences between sterile and non-sterile units were observed.

The piston keeps its characteristics after irradiation at a dose of 50 kGy.

### References

Design History File: 001-DHF-NIV

### Example 3

### Test Set-Up

### See FIG. 2

### Test Procedure

Take a Bone Substitute device assembly without luer cap.
Draw into the syringe a volume of at least 2 ml of freshly boiled water, cooled to room temperature.
With the screwing top female luer uppermost, withdraw the plunger axially until the fiducial line is at the nominal capacity graduation line. Clamp the plunger in this position using an appropriate fixture (RE.REF#189).
Connect the screwing cap female luer to the 3-way stopcock. Position the 3-way stopcock such that vacuum will be drawn in all directions.
Switch on the vacuum pump and allow the vacuum to stabilize. In the protocol 173-P-NIV it was asked to stabilize the pressure at 0.88 bar, however with the vacuum pump used the pressure was stabilized between 0.88 and 0.93 bar. During the stabilisation observe for air bubbles that break free from the piston seal. No more than 2 bubbles that break free are acceptable. If more than 2 bubbles break free, it is possible that air is being withdrawn from in-between piston seals. Record the location of leaks if any.
Position the 3-way stopcock such that the BSD and the pressure monitor are isolated from the vacuum pump. Turn off the vacuum pump and record the pressure read by the pressure manometer (initial pressure).
Start the stopwatch and allow the test sample to remain under vacuum for 60 (+5, −0) seconds.
During the hold period observe the piston seals for bubbles that form and break free. No replacing bubbles are acceptable.
At the completion of the hold period record the pressure read by the pressure manometer (final pressure). Examine the syringe to determine if the piston has become detached from the plunger. No vacuum decay or piston detachment is acceptable.
Remove the syringe from the 3-way stopcock.

### Example 4

### Test Set-Up

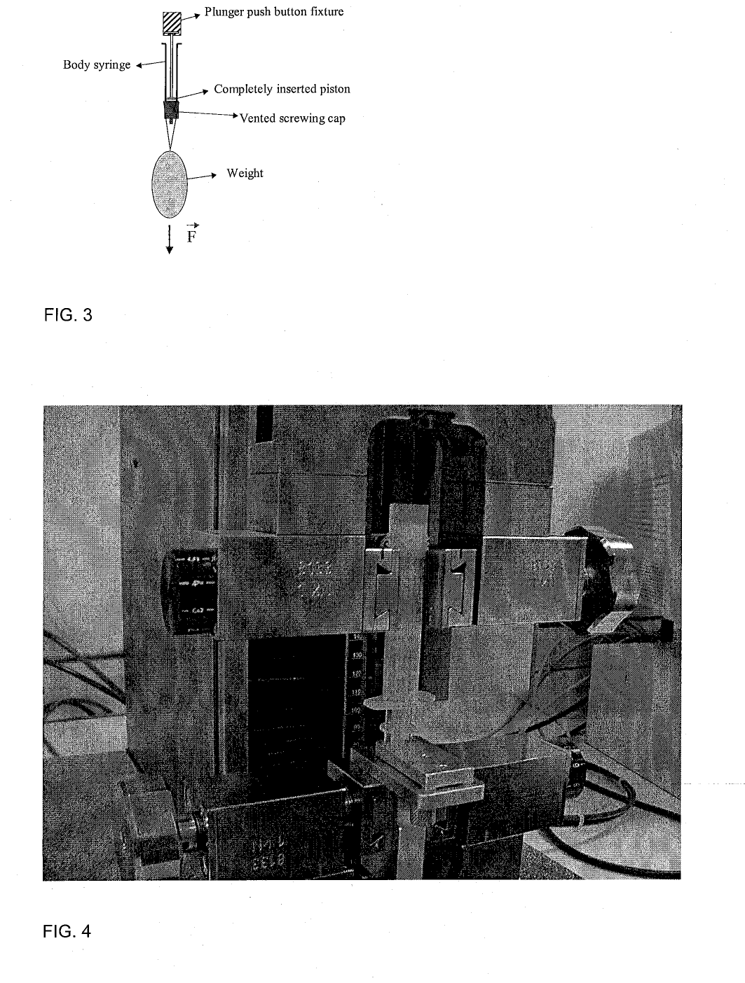

### See FIG. 3

Take a test unit (screwing cap/body syringe/plunger/piston assembly).
Put the piston to completely inserted position.
Check that the piston is fully inserted into body syringe and that it is firmly threaded into the plunger.
Place the weight on a firm flat surface.
Fix the weight to the screwing cap thanks to a suitable fixture (RE.REF#188).
Slide the plunger push button into the plunger fixture taking care not to move the piston inside the body syringe.
Pick up the weight and the test sample by the weight taking care not to move the piston inside the body syringe and release the weight and allow it to drop onto the landing area.
The body syringe/screwing cap assembly should remain attached to the weight as it is pulled off of the piston/plunger assembly.
Observe the piston/plunger assembly. If the piston remains attached to the plunger after the body syringe has been pulled off, the piston has passed the test.
If the piston detaches from the plunger, the piston has failed the test.

### Example 5

### Test Procedure

Take a test unit (body syringe/screwing cap/piston/plunger)
Put the piston to completely inserted position.
Check that the piston is fully inserted into body syringe and that it is firmly threaded into the plunger.
Fix the screwing cap in the upper jaw of a tensile machine thanks to a suitable fixture (RE.REF#190).
Fix the plunger push button in the lower jaw of a tensile machine thanks to a suitable fixture (RE.REF#190).
Zero the recorder and set the tensile machine so that it can apply a tensile force
Start the tensile machine so that it pulls the plunger/piston assembly till it is pull off of the body syringe with a crosshead speed of 500 mm/min.
Record the peak force when the plunger passes through the body syringe undercut.
The peak force should be higher than 29 N to pass the test and it is preferable that the peak force is higher than 59N.

### Test Set-Up

### See FIG. 4

### Example 6

### Test Set-Up

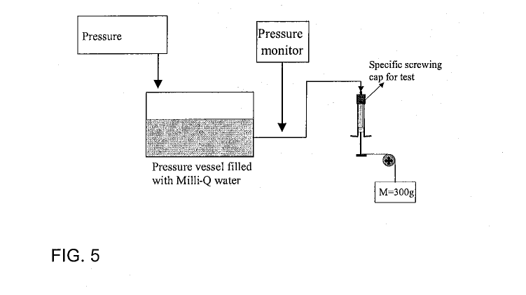

### See FIG. 5

### Test Procedure

Take a test unit (body syringe/piston/plunger assembly);
Screw the specific screwing cap for test;
Draw into the syringe a volume of water exceeding the nominal capacity of the syringe;
Expel air and adjust the volume of water in the syringe at normal capacity;
Seal the specific screwing cap for test with the water connection;
Fix the body syringe vertically with a specific fixture;
Apply a sideways force to the syringe to the plunger push button at right angle to the plunger to swing the plunger radially about the piston seal(s) with a force of about 3 N. During testing the set-up shown above with a weight of 300 g was not used, but the sideways force was applied by the hand of the person performing the test;
Orientate the plunger to permit the maximum deflection from the axial position;
Increase the water pressure till 300 kPa;
Record the pressure measured by the pressure monitor;
Maintain the pressure for 30 (+5, −0) seconds;
Turn off the water pressure and remove the test unit;
Examine the syringe for liquid leakage beyond the piston seals to the outside. If no liquid is found, the unit is acceptable.

### Example 7

### Test Procedure

For each device, set the piston at graduation 3.4 before sterilization (only sterile units are tested).
Take a Bone Substitute Device and remove the screwing cap and the luer cap.
Do not move the syringe plunger. Leave it at its initial setting.
Mount the test unit in the tensile machine as shown in the photo above.
Start the testing machine so that it pushes the plunger at a rate of 100 mm/min, until the piston is about 1 mm out of the syringe body.

1. A device comprising a combination of a polypropylene body with a resilient piston selected from a piston coated with a laminated layer of polytetrafluoroethylene resin film and a piston coated with a laminated layer tetrafluoroethylene-ethylene resin film.

2. The device according to claim 1 in which the body is a syringe body.

3. The device according to claim 2, wherein the syringe body is made of natural polypropylene Grade HD810MO.

4. The device according to claim 1 further comprising a plunger, a piston, a body syringe, a screwing top and a luer cap, wherein the piston is made of butyl rubber coated with tetrafluoroethylene polymer resin and the syringe body is made of natural polypropylene Grade HD810MO.

5. A syringe comprising:
a body composed of polypropylene; and
a piston disposed in the body and adapted to slide with respect to the body, wherein the piston comprises a laminated layer selected from polytetrafluoroethylene resin film and tetrafluoroethylene-ethylene resin film.

6. The syringe of claim 5 wherein the body comprises polypropylene grade HD810MO.

7. The syringe of claim 5 wherein the syringe does not include silicone oil.

8. The syringe of claim 5 wherein the piston is capable of sliding with respect to the body with a force of less than 25 N without the use of silicone oil in a test according to ISO 7886-1:1993.

9. A syringe comprising:
a hollow barrel body composed of polypropylene grade HD810MO; and
a piston disposed in the body and adapted to slide with respect to the body, wherein the piston comprises butyl rubber with a laminated layer comprising a tetrafluoroethylene-ethylene resin film.

10. The syringe of claim 9 wherein the syringe does not include silicone oil.

11. The syringe of claim 9 wherein the piston is capable of sliding with respect to the body with a force of less than 25 N without the use of silicone oil in a test according to ISO 7886-1:1993.
